(19) United States
(12) Patent Application Publication
(10) Pub. No.: US 2015/0335272 A1
(43) Pub. Date: Nov. 26, 2015

(54) BLOOD SUGAR CONCENTRATION (BSC) TESTING AND MONITORING SYSTEM AND METHOD

(71) Applicants: Nicholas R. Natale, Webster, NY (US); Nicholas P. Nicoletti, Rochester, NY (US)

(72) Inventors: Nicholas R. Natale, Webster, NY (US); Nicholas P. Nicoletti, Rochester, NY (US)

(21) Appl. No.: 14/717,366

(22) Filed: May 20, 2015

Related U.S. Application Data
(60) Provisional application No. 62/000,586, filed on May 20, 2014.
(60) Provisional application No. 62/121,271, filed on Feb. 26, 2015.

(51) Int. Cl. A61B 5/145; A61B 5/15; A61B 5/00

(52) U.S. Cl. CPC A61B 5/14532; A61B 5/742; A61B 5/14503; A61B 5/150358; A61B 5/0022

(57) ABSTRACT

Disclosed and depicted is a system and method for reliably testing, monitoring and predicting blood sugar concentration (e.g., glucose) for a user.

This application claims priority under 35 U.S.C. §119 to U.S. Provisional Application No. 62/000,586 for a BLOOD SUGAR CONCENTRATION (BSC) TESTING AND MONITORING SYSTEM AND METHOD, filed May 20, 2014 by Nicholas R. Natale et al., and U.S. Provisional Application No. 62/121,271 for a BLOOD SUGAR CONCENTRATION (BSC) TESTING AND MONITORING SYSTEM AND METHOD, filed Feb. 26, 2015 by Nicholas R. Natale et al., and both provisional applications, including any appendices filed therewith, are hereby incorporated by reference in their entirety.

Disclosed is a system, components of which may be located or work on a user's body (e.g., wrist), to provide improved access to blood glucose testing and to enable reliable monitoring and prediction based upon a user's activity level.

### BACKGROUND & SUMMARY

As used herein the term “blood sugar concentration” or “BSC” is intended to generally represent a characterization of blood sugar levels (e.g., glucose) that is accomplished through the collection and analysis of a sample, such as sampling accomplished using well-known test strips.

One aspect of the disclosed device is that it provides some functionality that is similar in nature to well-known glucose meters such as those depicted in FIG. 1. However, one of the disclosed embodiments, generally referred to herein as the BSC Band, is a novel device that provides diabetics or other needing to carefully monitor blood sugar levels with an alternative to the cumbersome “baggage” of traditional glucose meters, particularly in the form of a wearable device. The BSC Band is, in one embodiment disclosed herein, also suitable for use with a smart phone application, which provides the user with a variety of state of the art features.

Disclosed in embodiments herein are a system and method for reliably testing, monitoring and predicting BSC (glucose) for a user, comprising: a user-wearable apparatus (e.g., cuff or band), said apparatus including storage for glucose testing strips, a spring-loaded lancet, a strip reader, a display, an activity sensor (e.g., accelerometer), a processor and associated memory for collecting and storing, blood glucose levels based upon a test strip reading along with activity levels of the user based upon accelerometer output; a transceiver (e.g., Bluetooth) to permit the exchange (wired or wireless) of such data with a second computing device (e.g., smartphone); and a program, operating on either the wearable apparatus or the second computing device, to receive data representing the blood glucose levels along with activity levels of the user, and calculating a predicted blood glucose level for the user.

Also disclosed in embodiments herein is a system for reliably testing, monitoring and predicting blood sugar concentration for a user, comprising: a user-wearable apparatus, said apparatus including storage for blood sugar testing strips, a spring-loaded lancet, a strip reader, a display, an activity sensor, a processor and associated memory for collecting and storing, blood sugar levels based upon a test strip reading along with activity levels of the user based upon activity sensor output; a transceiver to exchange blood sugar and activity level data with a second computing device; and a program, stored in a memory and operating to receive the blood sugar and activity level data, and using said data calculating a predicted blood sugar level for the user.

Further disclosed herein is a method for predicting blood sugar concentration for a user, comprising: applying a user-wearable apparatus about a wrist of the user, said apparatus including storage for glucose testing strips, a spring-loaded lancet, a strip reader, a display, an activity sensor, a processor and associated memory; collecting and storing blood sugar concentration levels for the user based upon a test strip reading; monitoring activity levels of the user based upon output of the activity sensor, and calculating a predicted blood glucose level for the user; exchanging data, including blood sugar concentration and activity data, with a second computing device; and representing, on a display viewable by at least the user, blood sugar concentration and activity levels of the user.

Disclosed herein is a system for monitoring blood sugar concentration for a user, comprising: a user-wearable apparatus (e.g., cuff or band), said apparatus including storage for glucose testing strips, a spring-loaded lancet, a strip reader, a display, an activity sensor (e.g., accelerometer), a processor and associated memory for collecting and storing, blood glucose levels based upon a test strip reading along with activity levels of the user based upon accelerometer output; a transceiver (e.g., Bluetooth) to permit the exchange (wired or wireless) of such data with a second computing device (e.g., smartphone); a program, stored in a memory and operating on the second computing device, to receive data representing the blood glucose levels along with activity levels of the user, and calculating a predicted blood glucose level for the user; and a user interface, associated with the second computing device, said interface displaying at least one of: a measured blood glucose level, the blood glucose level for the user, an activity level for the user, said user interface further including a transparent drop menu providing a navigational shortcut to an interface page selected from the group consisting of: correction, mode, status, food and activity.

### BRIEF DESCRIPTION OF THE DRAWINGS

FIG. 1 is an illustration of various commercially available blood glucose meters;

FIG. 2 is a perspective view of an embodiment of the blood sugar concentration (BSC) testing and monitoring system;

FIG. 3 is an enlarged view of the operational portion of the system of FIG. 2;

FIGS. 4-6, illustrate various views of the modes in which the apparatus depicted may be employed;

FIG. 7 is a schematic illustration of the components employed within the user-wearable apparatus, and associated functionality of the apparatus;

FIG. 8 is an illustration of various views of an embodiment of the BSC band;

FIG. 9 illustrates the user-wearable apparatus paired via Bluetooth with a smart phone;

FIGS. 10-12 are illustrative representations of the functionality of the disclosed system operating with a paired device; and

FIGS. 13-20 are illustrative user interface screens for a smartphone application that interfaces with and to the blood sugar concentration testing and monitoring system.

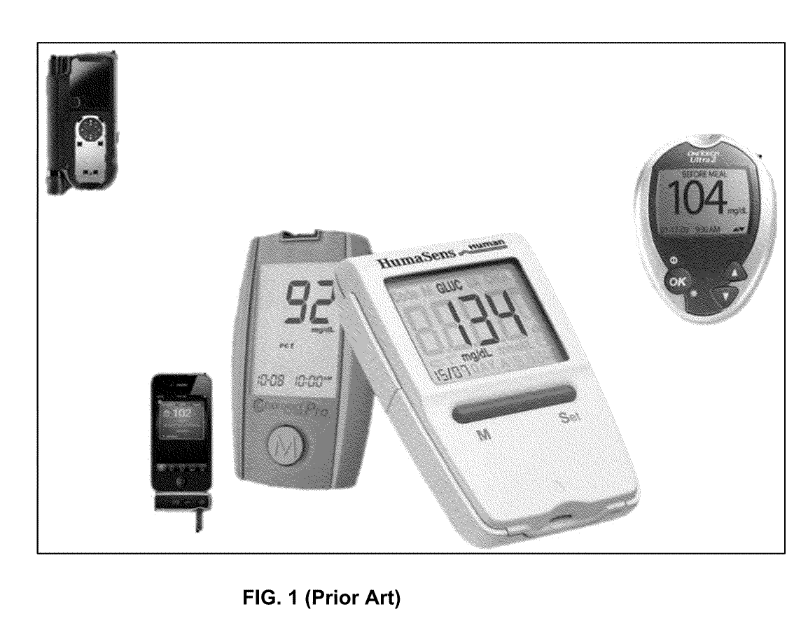

The various embodiments described herein are not intended to limit the disclosure to those embodiments described. On the contrary, the intent is to cover all alternatives, modifications, and equivalents as may be included within the spirit and scope of the various embodiments and equivalents set forth. For a general understanding, reference is made to the drawings as well as the Appendix incorporated herein. In the drawings, like references have been used throughout to designate identical or similar elements. It is also noted that the drawings may not have been drawn to scale and that certain regions may have been purposely drawn disproportionately so that the features and aspects could be properly depicted.

### DETAILED DESCRIPTION

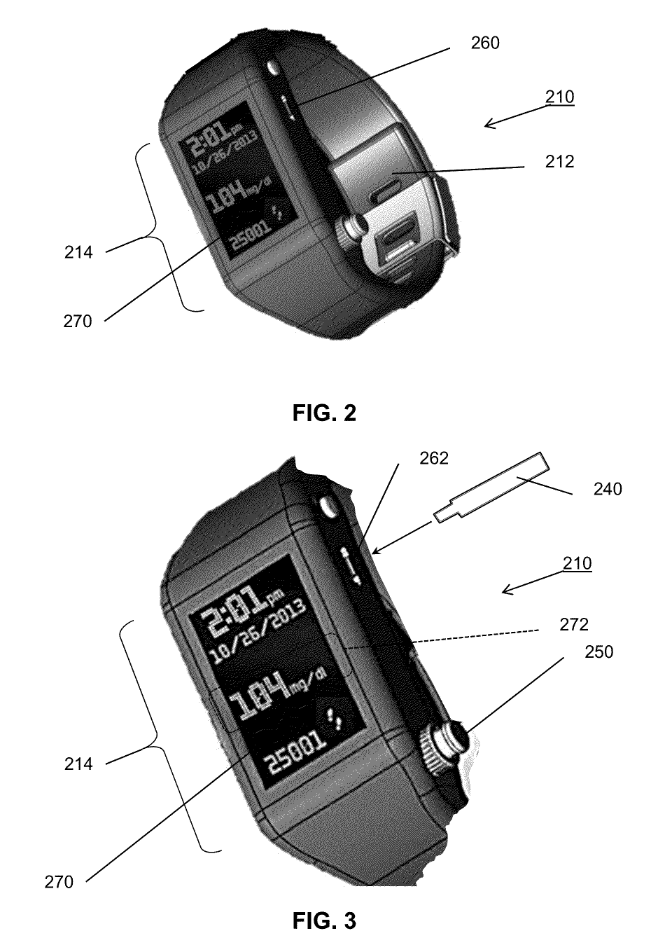

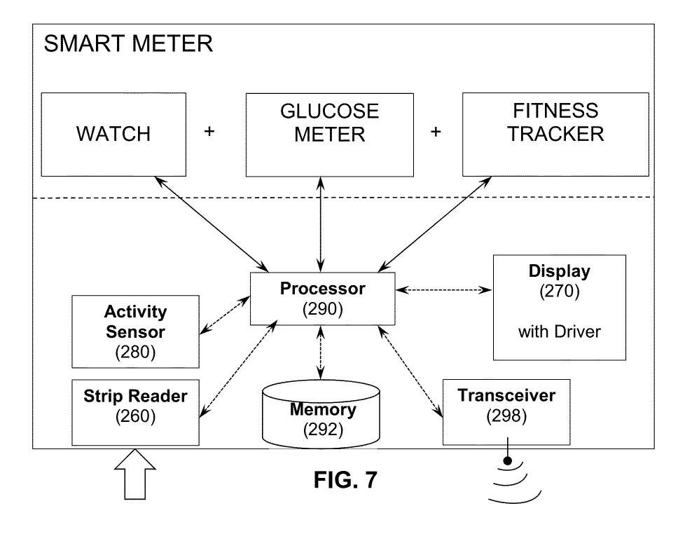

FIG. 2 is a perspective view of an embodiment of the BSC testing and monitoring system, and in particular a user-wearable apparatus 210, where the system is depicted as a cuff or band 212 that may be worn around a user's wrist (not shown) to hold an associated case 214 that houses a user-wearable apparatus providing various features as further described herein. It will also be appreciated that other configurations are possible, such as an arm-band around the upper arm, etc. The BSC band 210 of FIGS. 2-5 provides both a device and method for reliably testing, monitoring and predicting BSC (glucose) for a user. As further illustrated in the schematic of FIG. 7, for example, the BSC band comprises a user-wearable apparatus within a “case” attached to a cuff or band. The apparatus includes physical storage, such as a sliding tray or compartment 230, for glucose testing strips 240, a spring-loaded lancet 250, a strip reader 260, a display 270, an activity sensor 280 (e.g., accelerometer), a processor 290 and associated memory 292 for collecting and storing, among other data, blood glucose levels based upon a test strip reading.

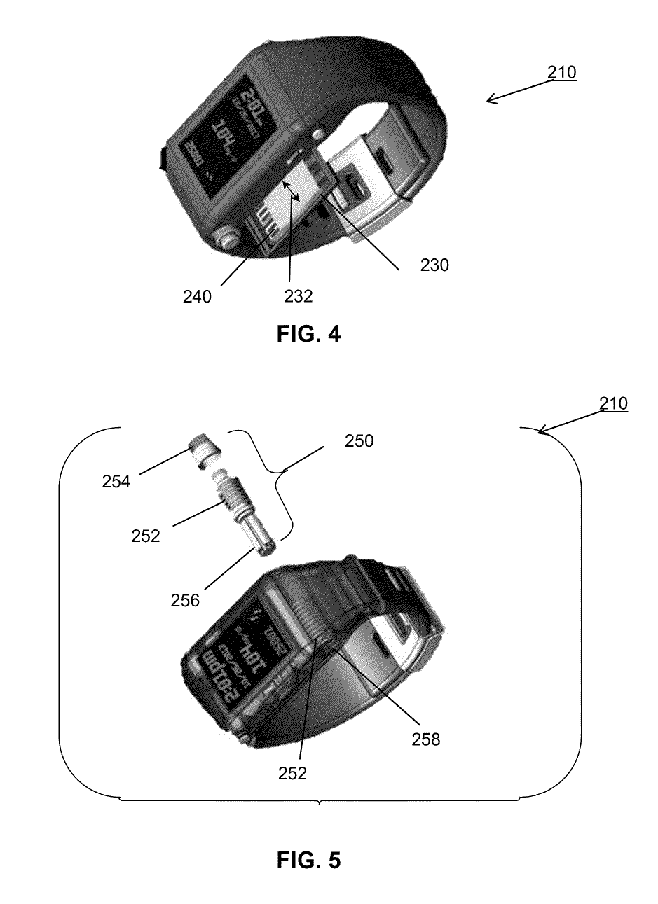

As represented, for example in FIGS. 4-8, the BSC band 210 may further include the ability to estimate or track a wearer's activity levels based upon the output of an activity sensor 280. In one embodiment the activity sensor may be an accelerometer. In alternative or additional embodiments, the activity sensor may receive and incorporate signals or data from a pedometer or a global positioning system (GPS) tracking system, etc., operatively associated with the apparatus. Moreover, as the apparatus 210 may be paired with a smartphone or similar device as disclosed below, it is further conceivable that a fitness app installed on the user's smartphone may be suitable to provide activity data for the user. Thus a smartphone may provide the functionality of the activity sensor in an alternative embodiment.

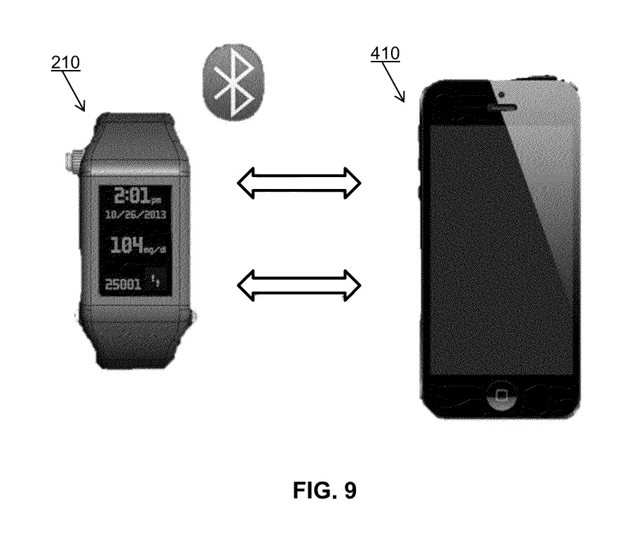

The illustrated band also includes a transceiver 298 (e.g., Bluetooth®, infrared, etc.) to permit the exchange (wired or wireless) of such data with a second computing device (e.g., smartphone). The functionality of the apparatus in association with the second computing device is further described below relative to FIGS. 9-20). And, a program(s) or application, stored in a memory and operating on either the user-wearable apparatus, or the second computing device, is suitable to receive data representing the blood sugar concentration levels along with activity levels of the user, and then calculate a predicted blood glucose level for the user.

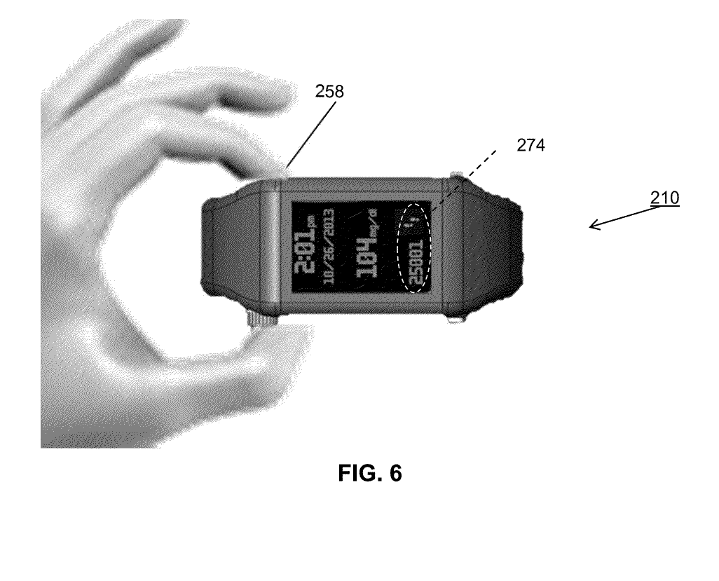

FIGS. 3-6, illustrate the various modes in which the disclosed apparatus depicted may be employed. For example, FIG. 4 illustrates test strips 240 stored in a slidable tray 230. Tray 230 may be stocked or replenished as necessary, and the user can manually open and close the tray by sliding it in or out from the case as indicated by arrow 232. Also considering FIG. 5, depicted therein is an assembly view of the spring-loaded (e.g., cock & release) lancet 250. As will be appreciated the lancet 250 includes springs 252, as well as a release mechanism that, under the power of a spring and in response to a user depressing button 254, temporarily extends the sharpened lancet tip (not shown) within sheath 256 to lance the tip of one of the user's digits adjacent aperture 258. In other words, FIG. 5 allows one-handed use of the device to pierce a finger or thumb pressed against the left side aperture 258 (see FIG. 6), followed by retraction and storage of the lancet. Once the lancet has been used, a test strip may be exposed to the user's blood and the exposed test strip is inserted into the side of the user-wearable apparatus for reading. Referring once again to FIG. 3, the test strip 240 is inserted into the aperture 262 in the side of the case, where the strip reader is positioned, to receive the strip and produce a reading of the user's blood sugar concentration. The reading produced is further illustrated as a reading on the display in region 272.

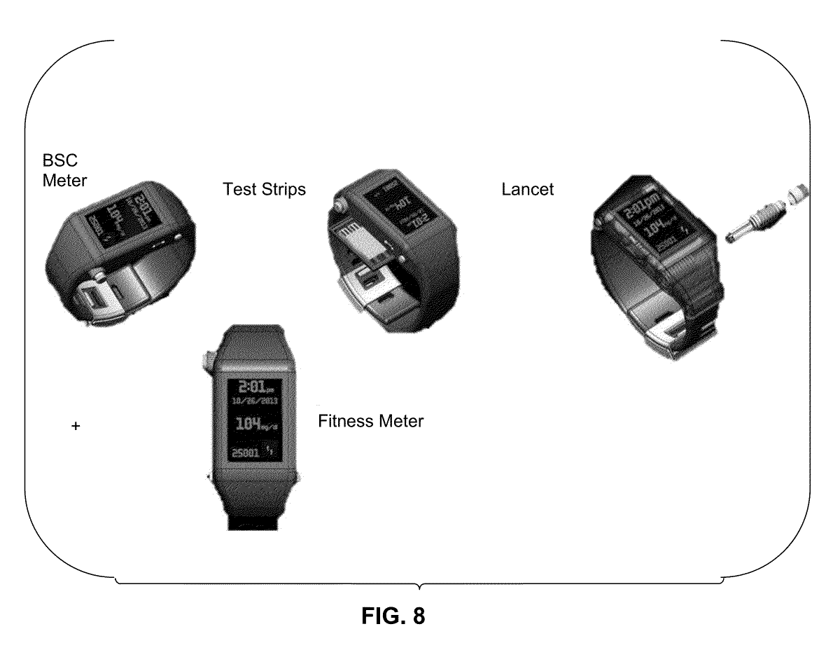

As illustrated in FIGS. 6 and 7, in one embodiment the user-wearable apparatus includes an activity sensor 280, such as an accelerometer incorporated therein, to track the user's activity. The activity sensor outputs the activity data to the processor, which in turn determines the activity to reflect (see e.g., footstep symbol and/or calorie counter in region 274 at bottom of the display), and the apparatus or smartphone app may be pre-programmed to exchange data representing the blood glucose levels along with activity levels of the user, as well as other information (e.g., weight, insulin administered, etc.), in order to calculate a predicted blood glucose level for the user. The various features and functions of the user-wearable apparatus are illustrated in the various views of an embodiment of the apparatus as illustrated in FIG. 8.

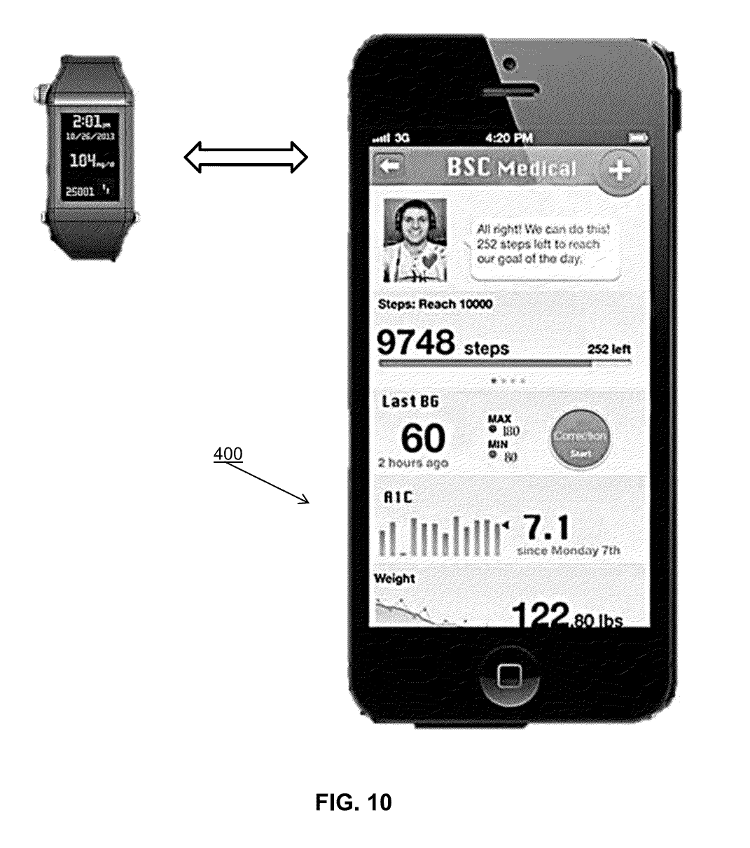

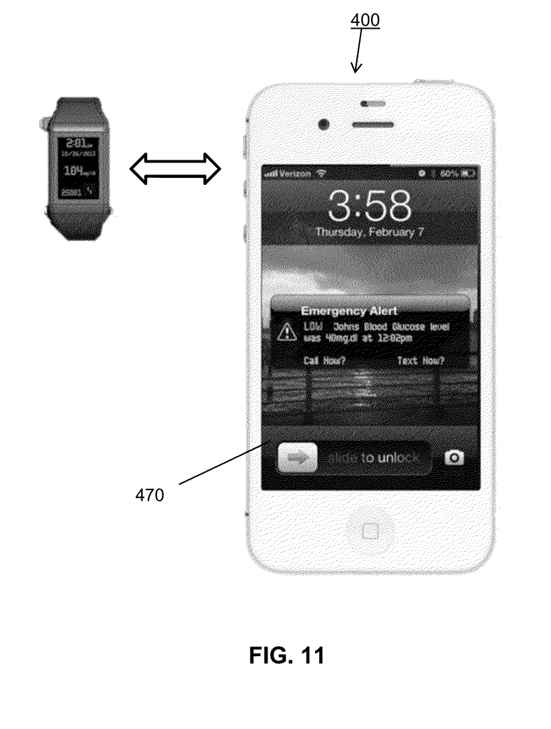

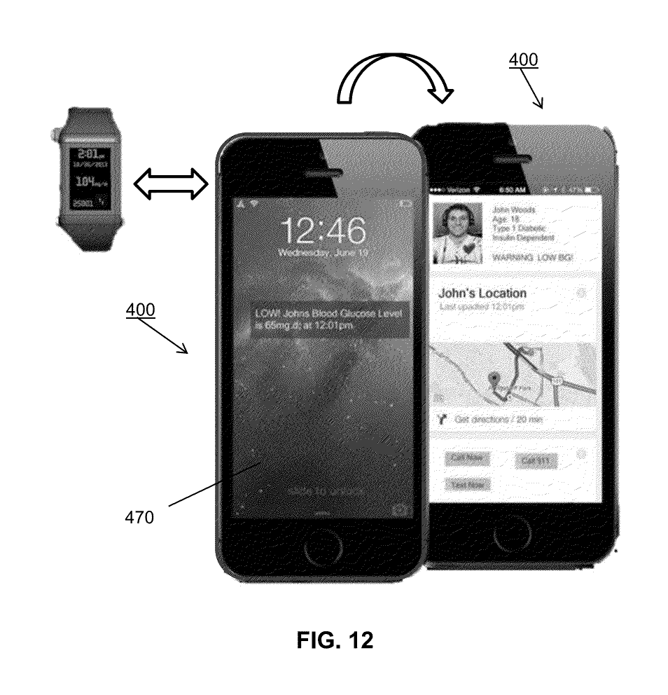

FIG. 9 illustrates that the user-wearable apparatus 210 may be paired via Bluetooth® or similar wireless communication techniques to a smartphone 410 or similar portable or stationary secondary computing and communication device. Such a pairing or interactive exchange of data between the devices permits data from the user-wearable apparatus to be shared, further processed, etc. As will now be described, the exchange of data facilitates a number of features. FIGS. 10-13 are illustrative representations of some of the functionality of an alternative embodiment of the system (e.g., user-wearable apparatus 210 in combination with a secondary computing device(s) 400 such as smartphone), where an application (app) on the smartphone may depict data for the user (e.g., FIG. 10) on display 470, provide a warning when tested or predicted blood sugar concentration is out of range (e.g., too low as shown in FIG. 11), and even initiate contact with another communication device or a central hotline, etc. to report the position of the user in the event of a warning or alarm condition (FIG. 12).

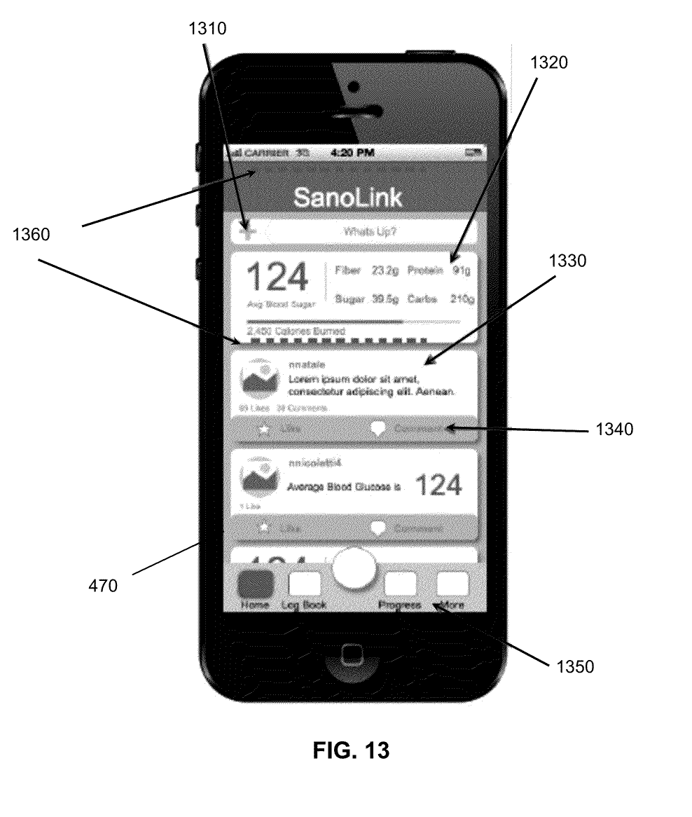

Having generally described embodiments of the BSC band and its functionality, discussion is now directed to the user interface associated with a related smartphone application as illustrated in FIGS. 13-19. And, while noting that the application may be carried out on the smartphone, it is also conceivable that the functions described may, in some or all cases, also be accomplished on an embodiment of the user-wearable apparatus as well. In general, the user interface depicted is associated with a second computing device (410) such as a smartphone, although it is entirely conceivable that the functionality of the user interface could, at least in part, be incorporated within the BSC band (210) itself. The user interface has a display for displaying at least one of the following: a measured blood glucose level, the blood glucose level for the user, an activity level for the user. The user interface may further include a transparent drop menu providing a navigational shortcut to an interface page selected from the following: correction, mode, status, food and activity.

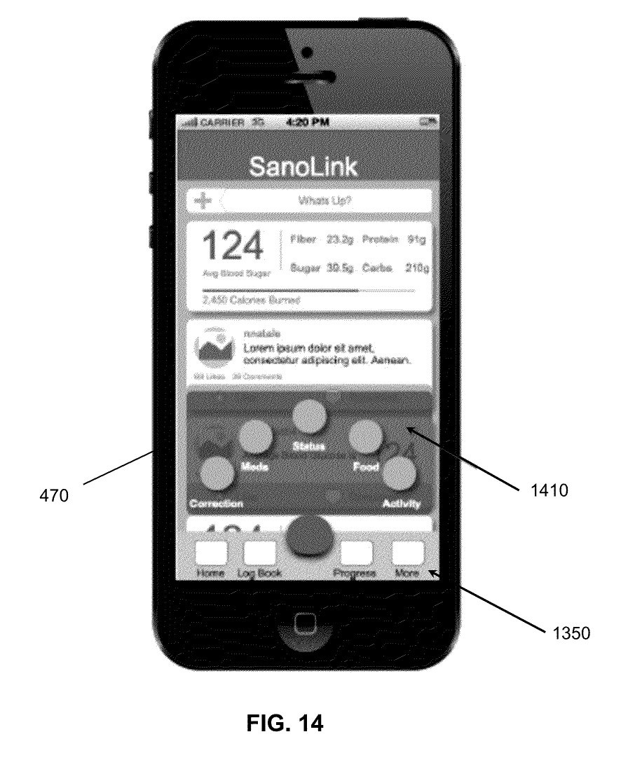

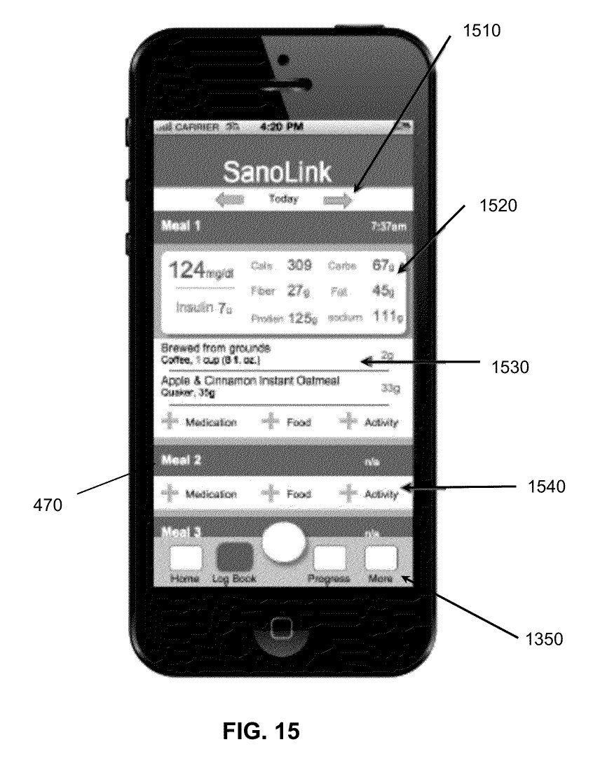

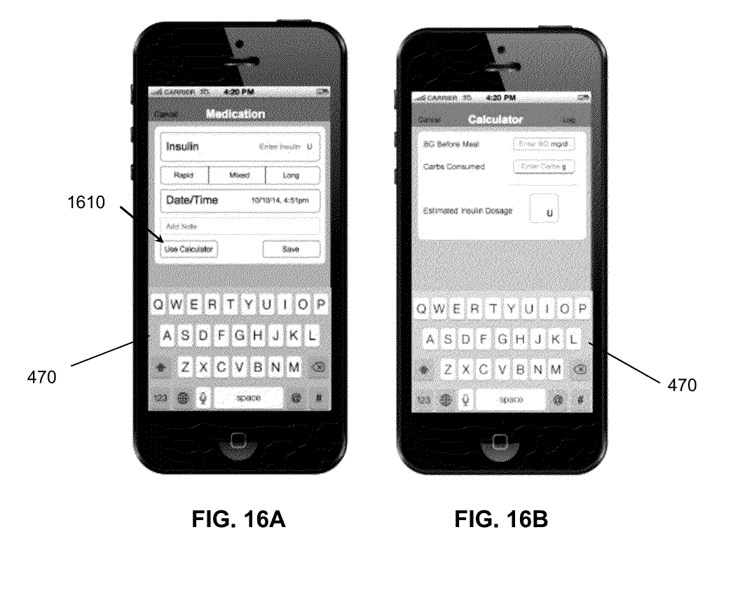

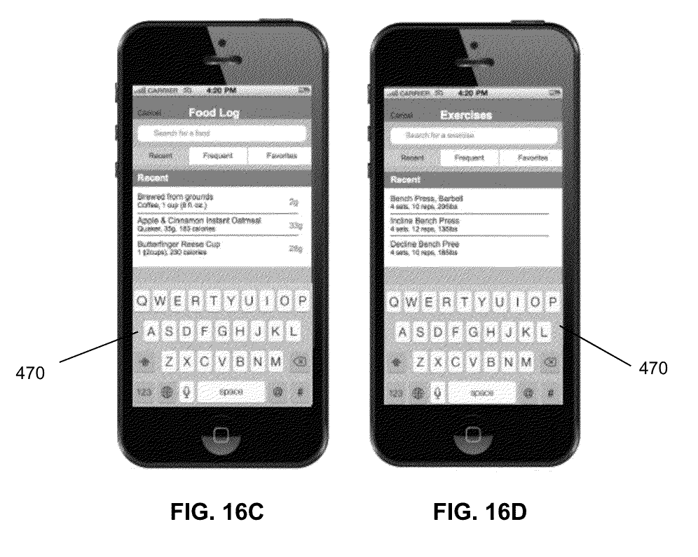

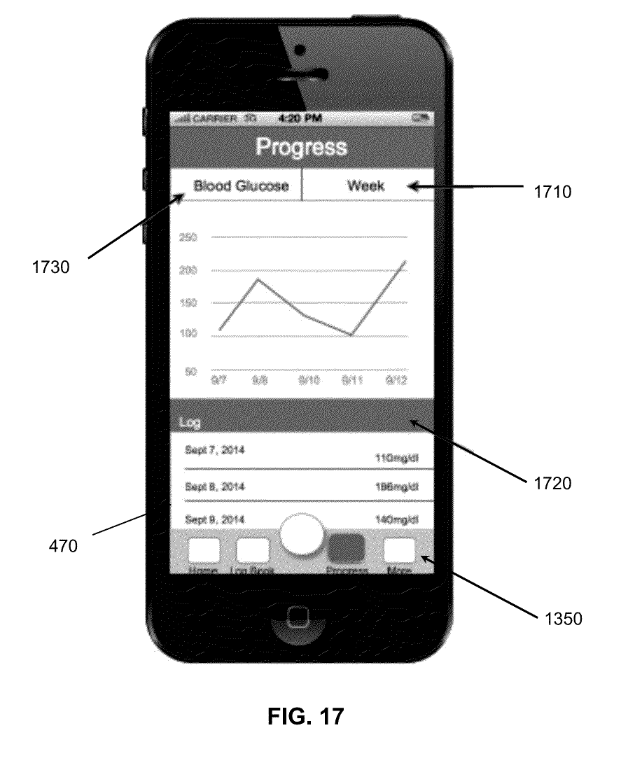

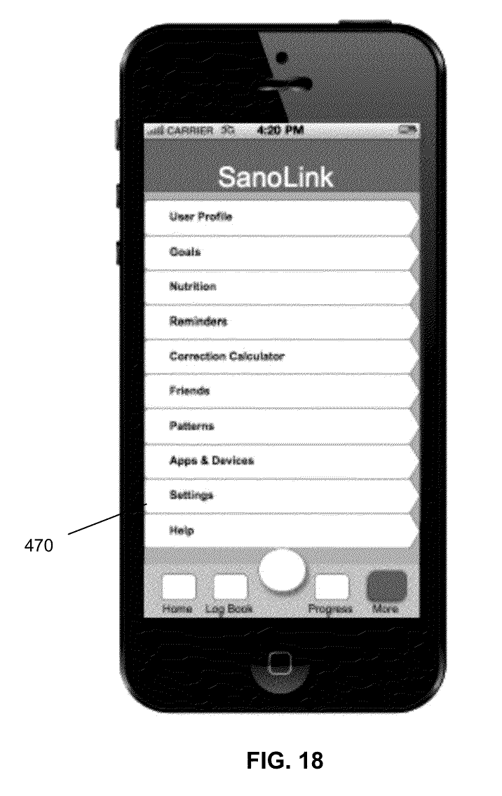

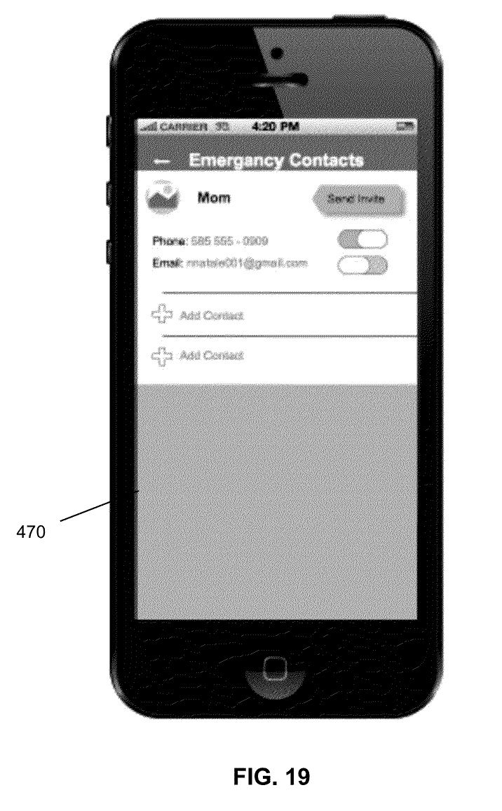

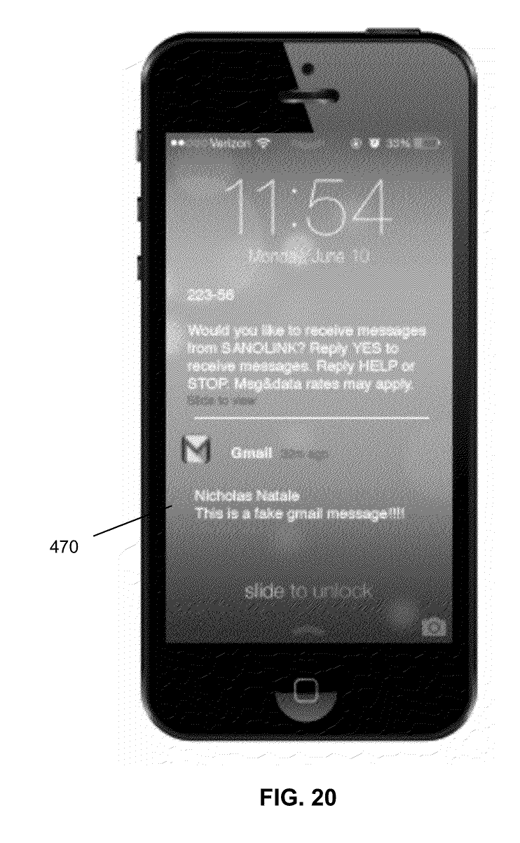

Referring to FIGS. 13-20, the application user interface further includes at least one display element such as a navigation link, button, etc. The navigation features, as illustrated in the figures, such as the home page of FIG. 13, may be selected from one or more of the following:
a forward and backward navigational aid, such as illustrated in FIG. 14, to see data chronologically in advance of and behind the currently displayed data;
a nutritional summary page (FIG. 15, log book), which may also include a nutritional summary providing totals (e.g., carbs) for all nutrition logged during a displayed time period (e.g., meal or day), and a detailed listing of nutrition logged as having been ingested during a displayed time period;
a medication summary page (FIG. 16A), that may further include a listing of medications ingested (past) or anticipated (future) during a displayed time period (e.g., day), and/or a field for notes related to the medication;
a calculator, for example as illustrated in FIG. 16B, to assist the user in determining an appropriate medication dosage, said calculator receiving inputs such as a blood glucose level measured before the user took on nutrition (e.g., a meal, as represented in FIG. 16C) and the carbs logged for the nutrition and exercise or activity (FIG. 16D) and outputting an estimated medication dosage (e.g., insulin units);
a progress page such as FIG. 17, which may also include a graphical representation of the logged blood glucose readings over a selected period of time (e.g., a week), and a scrollable table listing the data displayed in the graphical representation
a user page (FIG. 18) displaying navigation links, including: user profile, goals, nutrition, reminders, correction calculator, friends, patterns, applications and devices, settings and help; and
an emergency contacts listing such as illustrated in FIG. 19 including a link to “invite” the contact to receive alerts as illustrated in FIG. 20 (e.g., via text or e-mail).

More specifically, referring to FIG. 13, the interface 470 includes a status widget 1310 that allows users to easily share their thoughts, questions, concerns, and motivational stories with their own personal diabetic community (e.g., family and friends). The user profile widget in region 1320 would be displayed at the top of the user's own feed, and no one would see this, but the user themselves. If the user chooses to share this information with friends they could then post the information into the feed. An example of a user status, which would be posted to a “wall”, is illustrated in region 1330. Status would be a continuous thread, and users would also be able to share their current status of wellness throughout the day. In region 1340, other users would be able to “comment” and “like” user A's posting as the post appears on their feed. The navigation bar in region 1350 would be provided on every page of the application. These buttons would link to other pages within the application. The navigation bar is the only static image on the display page. As represented by dashed lines 1360, as the user scrolls in the interface these features would collapse into the display page.

Depicted in FIG. 14 is a representation of an exemplary navigation display page, where the center circle button would present to the user a transparent drop menu 1410. This menu could be used as a shortcut tool from any display page. The user could quickly use the correction calculator, add medication taken, write a post, log food and activity, etc. Also referring to the display of FIG. 15, arrows 1510 can be used to navigate—for example backward and forward between dates in the log book timeline. In region 1520 is a widget that totals out the primary nutrition facts for all the foods logged for a meal. Also displayed is the blood sugar before the meal and the insulin taken. In region 1530, below the meals widget is a list of the foods logged for that meal. To the right of each entry are the carbohydrates (carbs) for each food. In region 1540 on the display page the tools bring the user to another page in which they could manually input medication, food, and activity—where the activity would sync automatically based upon the data out from the user-wearable apparatus.

Selection of the “LogBook” button in region 1350 of the menu results in the display of pages such as those illustrated in FIGS. 16 A -D. FIGS. 16 A-D illustrate various features of the Log Book functionality of the application, whereby a user can record or log information via the interface; information that is stored, at least temporarily, in the memory of the device for subsequent access. Briefly, FIG. 16 A illustrates a medication logging feature whereby the type of medication and the dosage can be recorded. The “Use Calculator” button (1610) results in the display of a dosage calculator feature as represented in the interface display page of FIG. 16B. Similarly, the log pages of FIGS. 16C and 16D, respectively, provide a user with food and exercise information. Each display page not only shows prior log information that was entered in the middle of the display page, but provides a field at the top of the page to allow the user to enter additional log information, and/or edit existing information, via a keypad at the bottom of the interface.

Referring to FIG. 17, based upon stored data, the Progress display page is presented in response to a user selecting the “Progress” button in the navigation menu region 1350. In response the system is able to provide the user with information illustrating how well the user has managed his/her own blood sugar concentration during a prior period of time. In the illustrated embodiment the data is not only represented in a table (1720), but also as a graphical representation at the top of the user interface display page. Region 1710 allows users to switch the timeline being displayed from day, week, month, and year. In a similar manner, selection within region 1730 allows users to switch the data being displayed between Blood Glucose, Calories, Carbs, and Steps or a similar exercise metric (e.g., mets).

In FIG. 18, display 470 provides an interface to other features of the application, including information, social media links, settings, etc. Under at least the “Friends” link, it is possible to add and edit people from your contacts list. For example, referring to FIG. 19, “Mom” could be set as an emergency contact. When selected as an emergency contact, “Mom” would be notified via a SMS prompt to accept or approve the request to be added as a contact. As an example, “Mom” might receive a communication as illustrated in FIG. 20. After an emergency contact is added, the user could send an invitation to receive alerts via text or email. FIG. 20 presents an exemplary display page indicating how “Mom” would subscribe.

It should be understood that various changes and modifications to the embodiments described herein will be apparent to those skilled in the art. Such changes and modifications can be made without departing from the spirit and scope of the present disclosure and without diminishing its intended advantages. It is therefore anticipated that all such changes and modifications be covered by the instant application.

What is claimed is:

1. A system for reliably testing, monitoring and predicting blood sugar concentration for a user, comprising:
a user-wearable apparatus, said apparatus including storage for blood sugar testing strips, a spring-loaded lancet, a strip reader, a display, an activity sensor, a processor and associated memory for collecting and storing, blood sugar levels based upon a test strip reading along with activity levels of the user based upon activity sensor output;
a transceiver to exchange blood sugar and activity level data with a second computing device; and
a program, stored in a memory and operating to receive the blood sugar and activity level data, and using said data calculating a predicted blood sugar level for the user.

2. The system according to claim 1, wherein a display is presented to the user depicting the blood sugar level as well as a warning when the predicted blood sugar level for the user is outside an acceptable range set for the user.

3. The system according to claim 2, further including a secondary communications channel wherein the warning that the predicted blood sugar level for the user is outside an acceptable range is further communicated to a second device.

4. The system according to claim 3, wherein said secondary communication channel is selected from the group consisting of text/SMS messaging, telephone signal, and e-mail.

5. The system according to claim 1, wherein said spring-loaded lancet includes a mechanism that enables one-handed use of the device to, in response to a user depressing one side of the user-wearable apparatus, briefly extend and then retract a lancet tip to pierce a digit of the hand pressed against the user-wearable apparatus, thereby enabling the user to expose a testing strip to a lanced location on the digit.

6. The system according to claim 5, wherein said strip reader is located on a side of the user-wearable apparatus, and an exposed test strip is inserted into the strip reader for reading, said strip reader operating to generate a blood sugar level reading for storage in the associated memory.

7. A method for predicting blood sugar concentration for a user, comprising:
applying a user-wearable apparatus about a wrist of the user, said apparatus including storage for glucose testing strips, a spring-loaded lancet, a strip reader, a display, an activity sensor, a processor and associated memory;
collecting and storing blood sugar concentration levels for the user based upon a test strip reading;
monitoring activity levels of the user based upon output of the activity sensor, and calculating a predicted blood glucose level for the user;
exchanging data, including blood sugar concentration and activity data, with a second computing device; and
representing, on a display viewable by at least the user, blood sugar concentration and activity levels of the user.

8. The method according to claim 7, further including presenting, to the user, a display depicting the blood sugar level as well as a warning when the predicted blood sugar level for the user is outside an acceptable range.

9. The method according to claim 8, further including communicating the warning that the predicted blood sugar level for the user is outside an acceptable range to a second device.

10. The method according to claim 9, wherein said secondary communication channel is selected from the group consisting of text/SMS messaging, telephone signal, and e-mail.

11. The method according to claim 7, wherein said spring-loaded lancet permits one-handed use of the lancet to, in response to a user depressing one side of the user-wearable apparatus, briefly extend and then retract a lancet tip to pierce a digit of the hand pressed against the user-wearable apparatus.

12. The method according to claim 11, further including the lancet enabling the user to expose a testing strip to a lanced location on the digit.

13. The method according to claim 11, wherein said strip reader is located on a side of the user-wearable apparatus, and where an exposed test strip is inserted into the strip reader for reading, said strip reader operating to generate a blood sugar level reading for storage in the associated memory.

14. A system for monitoring blood sugar concentration for a user, comprising:
a user-wearable apparatus (e.g., cuff or band), said apparatus including storage for glucose testing strips, a spring-loaded lancet, a strip reader, a display, an activity sensor (e.g., accelerometer), a processor and associated memory for collecting and storing, blood glucose levels based upon a test strip reading along with activity levels of the user based upon accelerometer output;
a transceiver (e.g., Bluetooth) to permit the exchange (wired or wireless) of such data with a second computing device (e.g., smartphone);
a program, stored in a memory and operating on the second computing device, to receive data representing the blood glucose levels along with activity levels of the user, and calculating a predicted blood glucose level for the user; and
a user interface, associated with the second computing device, said interface displaying at least one of: a measured blood glucose level, the blood glucose level for the user, an activity level for the user, said user interface further including a transparent drop menu providing a navigational shortcut to an interface page selected from the group consisting of: correction, mode, status, food and activity.

15. The system according to claim 14, wherein said user interface further includes at least one display selected from the group consisting of:
i) a forward and backward navigational aid to see data chronologically;
ii) a nutritional summary page, comprising a nutritional summary for all nutrition logged during a time period, and a detailed listing of nutrition ingested during the time period;
iii) a medication summary page, comprising a listing of medications ingested or anticipated during a time period, and a field for notes related to the medication;
iv) a calculator to assist a user in determining an appropriate medication dosage, said calculator receiving, as input, a measured blood sugar concentration level before taking on nutrition and carbohydrates logged for the nutrition, and in response outputting an estimated medication dosage;
v) a progress page, comprising a graphical representation of logged blood sugar concentration readings over a time period, and a scrollable table listing the data displayed in the graphical representation;
v) a user page displaying navigation links, including user profile, goals, nutrition, reminders, correction calculator, friends, patterns, apps & Devices, settings and help; and
vi) a listing for at least one emergency contact, including a link to “invite” the contact to receive alerts.
